(19) United States
(12) Patent Application Publication
(10) Pub. No.: US 2025/0118413 A1
(43) Pub. Date: Apr. 10, 2025

(54) MEDICAL ASSISTANCE DEVICE

(71) Applicant: NIHON KOHDEN CORPORATION, Tokyo (JP)

(72) Inventor: Kou ADACHI, Saitama (JP)

(21) Appl. No.: 18/897,140

(22) Filed: Sep. 26, 2024

(30) Foreign Application Priority Data
Oct. 6, 2023 (JP) 2023-174481

(51) Int. Cl. G16H 20/40; G16H 20/10; G16H 70/40

(52) U.S. Cl. CPC G16H 20/40; G16H 20/10; G16H 70/40

(57) ABSTRACT

A medical assistance device includes at least one processor and at least one memory that stores at least one instruction executable by the processor, When the at least one instruction is executed by the processor, the medical assistance device is configured to: obtain past surgery information; generate anticipated surgery schedule information and anticipated auxiliary information; and generate display data. The past surgery information includes past required total time information on a previously performed surgery and past auxiliary information. The past auxiliary information includes at least one of medication information, and transition information. The anticipated auxiliary information includes at least one of anticipated medication information and anticipated transition information, based on the past surgery information. The display data includes the anticipated surgery schedule information and the anticipated auxiliary information.

### CROSS-REFERENCE TO RELATED APPLICATIONS

This application is based on and claims priority under 35 USC 119 from Japanese Patent Application No. 2023-174481 filed on Oct. 6, 2023, the contents of which are incorporated herein by reference.

### TECHNICAL FIELD The presently disclosed subject matter relates to a medical assistance device.

### BACKGROUND ART

JP2023-002479A discloses a surgical assistance system that displays a surgery schedule screen, on which real time surgery information is associated with a surgery schedule, on a monitor.

Surgery requires advanced medical techniques, and various procedures may be needed during a surgery. For this reason, a certain degree of experience may be required for a medical worker to appropriately perform a surgery. Although it is conceivable to determine in advance a surgery schedule, there is also a need to enable a less experienced medical worker to appropriately perform a surgery.

### SUMMARY

An object of the presently disclosed subject matter is to provide a medical assistance device that can assist even a less experienced medical worker to appropriately perform a surgery.

To achieve the above object, a medical assistance device according to an aspect includes:
at least one processor; and
at least one memory that stores at least one instruction executable by the processor, in which
when the at least one instruction is executed by the processor, the medical assistance device is configured to:
obtain past surgery information, the past surgery information including past required total time information on a previously performed surgery and past auxiliary information, the past auxiliary information including at least one of medication information, the medication information including at least timing information indicating a timing of medicating a patient in the previously performed surgery, and transition information indicating a transition of patient physiological information in the previously performed surgery;
generate anticipated surgery schedule information and anticipated auxiliary information, the anticipated auxiliary information including at least one of anticipated medication information and anticipated transition information, based on the past surgery information; and
generate display data including the anticipated surgery schedule information and the anticipated auxiliary information.

The medical assistance device having the above-described configuration generates the display data including the anticipated surgery schedule information and the anticipated auxiliary information, the anticipated auxiliary information including at least one of the anticipated medication information and the anticipated transition information, based on the past surgery information including the past required total time information and the past auxiliary information including at least one of the medication information and the transition information. For this reason, a medical worker can recognize not only the anticipated surgery schedule information but also at least one of the anticipated medication information and the anticipated transition information by visually checking the display data. Accordingly, the medical assistance device having the above configuration can assist even a less experienced medical worker to appropriately perform a surgery.

According to the presently disclosed subject matter, it is possible to provide a medical assistance device that can assist even a less experienced medical worker to appropriately perform a surgery.

### BRIEF DESCRIPTION OF DRAWINGS

FIG. 1 is a schematic diagram illustrating a medical assistance system according to an embodiment (hereinafter also referred to as the present embodiment) of the presently disclosed subject matter.

FIG. 2 illustrates an example of past surgery information.

FIG. 3 illustrates an example of patient information.

FIG. 4 illustrates an example of medical worker information.

FIG. 5 is a flowchart illustrating a medical assistance method according to a first example.

FIG. 6 illustrates an example of an image displaying anticipated procedure schedule information.

FIG. 7 illustrates an example of an image displaying anticipated medication information.

FIG. 8 illustrates an example of an image displaying sub-anticipated surgery schedule information on each medical worker.

FIG. 9 is a flowchart illustrating a medical assistance method according to a second example.

FIG. 10 illustrates an example of an image displaying updated anticipated procedure schedule information.

FIG. 11 illustrates an example of an image displaying updated sub-anticipated surgery schedule information.

FIG. 12 is a flowchart illustrating a notification method according to a third example.

### DESCRIPTION OF EMBODIMENTS

Hereinafter, an embodiment of the presently disclosed subject matter will be described with reference to the drawings.

### Overall Configuration

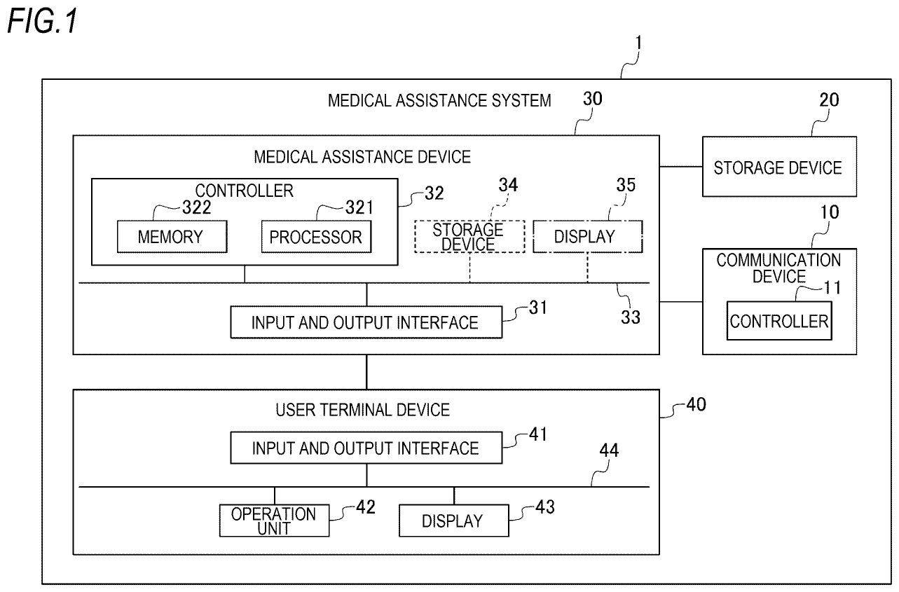

A medical assistance system 1 according to the present embodiment will be described with reference to FIGS. 1 to 4. FIG. 1 is a schematic diagram illustrating the medical assistance system 1 according to the embodiment of the presently disclosed subject matter. The medical assistance system 1 is used in a medical facility such as a hospital. In the present embodiment, it is assumed that percutaneous catheter ablation (example of a surgery) is previously performed a plurality of times at the medical facility. It is further assumed that percutaneous catheter ablation is currently being performed at the medical facility. In the present embodiment, current time is assumed to be Aug. 1, 2023. As illustrated in FIG. 1, the medical assistance system 1 can include a communication device 10, a storage device 20, a medical assistance device 30, and a user terminal device 40.

The communication device 10 is communicably connected to the medical assistance device 30 in a wired or wireless manner. The communication device 10 is a medical device used in a surgery or the like, for example, a medical camera, a medical robot, an information device that records an electronic medical record, and an electric scalpel. The communication device 10 can include a controller 11.

The controller 11 has a hardware configuration including a memory and a processor. The memory can include, for example, a read only memory (ROM) that stores various computer programs and the like, and a random access memory (RAM) having a plurality of work areas in which various computer programs to be executed by the processor are stored. The processor is, for example, a central processing unit (CPU), which loads a specified computer program from the various computer programs incorporated in the ROM onto the RAM and executes various processes in cooperation with the RAM.

The controller 11 obtains current procedure information on each of a plurality of procedures included in the currently performed surgery. For example, when the communication device 10 is a medical camera, a medical robot, or an information device that records an electronic medical record, the controller 11 obtains image data and video data captured during the surgery, medication data, and patient physiological information as examples of the current procedure information. For example, when the communication device 10 is an electric scalpel, the controller 11 obtains information on ON/OFF of a power supply (that is, information indicating whether the electronic scalpel is being used) or the like as the current procedure information. The controller 11 transmits the obtained current procedure information to the medical assistance device 30.

The plurality of procedures included in the surgery can include various actions for performing the surgery. For example, the plurality of procedures included in the surgery can include an action incidental to the surgery, such as entering the room, and a medical action such as anesthesia induction. The plurality of procedures included in the surgery are preset for each surgery. The plurality of procedures included in the surgery may also be freely changed by a medical worker or the like. For example, when the surgery is percutaneous catheter ablation, procedures included in the surgery can include entering the room, anesthesia induction, timeout, puncture, Brockenbrough, cauterization, left pulmonary vein (LPV) ablation, right pulmonary vein (RPV) ablation, induction, sheath removal, and exiting the room.

The current procedure information can include current medication information on medicating a patient in a procedure included in the currently performed surgery and current transition information indicating a transition of patient physiological information in a procedure included in the currently performed surgery. The controller 11 may transmit the obtained current procedure information to the medical assistance device 30.

The controller 11 may generate progress information indicating a progress of a procedure included in the currently performed surgery based on the current procedure information. In this case, for example, the controller 11 analyzes image data, video data, and the like captured during the surgery to determine whether the procedure included in the currently performed surgery is completed, and generates progress information based on the determination. When the progress information is generated, the controller 11 transmits the generated progress information to the medical assistance device 30 at, for example, a timing when the procedure included in the currently performed surgery is completed. The controller 11 may transmit the generated progress information to the medical assistance device 30 not only at the timing when the procedure included in the currently performed surgery is completed, but also at another timing. That is, the controller 11 transmits the generated progress information 10 to the medical assistance device 30 at least at the timing when the procedure included in the currently performed surgery is completed.

The storage device 20 stores, for example, various data. The storage device 20 is a storage such as a hard disk drive (HDD), a solid state drive (SSD), and a flash memory. The storage device 20 stores past surgery information, patient information, medical worker information, and the like.

The past surgery information is information on a previously performed surgery at a medical facility that uses the medical assistance system 1. The past surgery information can include surgery date information, surgery name information, related medical worker information, target patient information, past procedure information on each of a plurality of procedures included in the previously performed surgery, past required total time information on the previously performed surgery, and past auxiliary information.

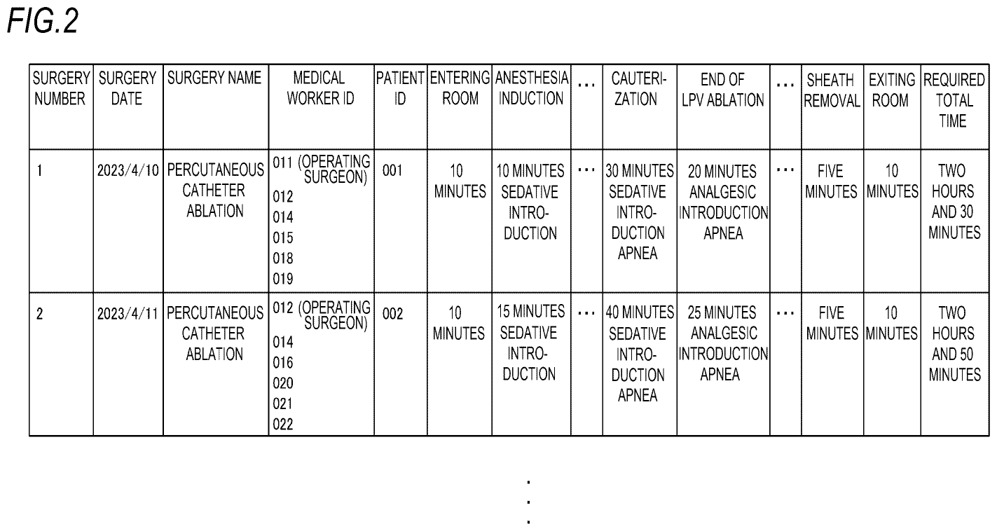

The surgery date information is information indicating a date on which the surgery is previously performed. For example, a date on which a surgery of a surgery number 1 illustrated in FIG. 2 is performed is Apr. 10, 2023.

The surgery name information is information indicating a name of the previously performed surgery. For example, a name of the surgery of the surgery number 1 illustrated in FIG. 2 is percutaneous catheter ablation.

The related medical worker information is information indicating a medical worker involved in the previously performed surgery. For example, medical workers involved in the surgery of the surgery number 1 illustrated in FIG. 2 are medical workers whose medical worker IDs are 011, 012, 014, 015, 018, and 019. The related medical worker information is not limited to the medical worker IDs, and may be medical worker names or the like. The related medical worker information can include operating surgeon information indicating an operating surgeon in the surgery. For example, an operating surgeon in the surgery of the surgery number 1 illustrated in FIG. 2 is a medical worker whose medical worker ID is 011.

The target patient information is information indicating a patient in the previously performed surgery. For example, a patient in the surgery of the surgery number 1 illustrated in FIG. 2 is a patient whose patient ID is 001. The target patient information is not limited to the patient ID, and may be a patient name or the like.

The past procedure information can include past required time information and past order information on each of a plurality of procedures included in the previously performed surgery. The past required time information is information indicating time required for a procedure included in the previously performed surgery. For example, the past required time information on a procedure of anesthesia induction in the surgery of the surgery number 1 illustrated in FIG. 2 is information indicating that time required for the procedure is 10 minutes. The past order information is information indicating an order of a procedure in a surgery including a plurality of procedures. For example, the past order information on the procedure of anesthesia induction in the surgery of the surgery number 1 illustrated in FIG. 2 is information indicating that an order of the procedure is a second procedure in the entire surgery.

The past required total time information is information indicating time required for the entire previously performed surgery. For example, the past required total time information in the surgery of the surgery number 1 illustrated in FIG. 2 is information indicating that time required for the surgery of the surgery number 1 is two hours 30 minutes.

The past auxiliary information can include medication information on medicating the patient in the previously performed surgery and transition information indicating a transition of patient physiological information in the previously performed surgery. The medication information can include timing information, content information, dose information, and ingredient information. The timing information is information indicating a timing of medicating the patient in the previously performed surgery. The content information is information indicating a content of medicine medicated to the patient in the previously performed surgery. The dose information is information indicating a dose of the medicine medicated to the patient in the previously performed surgery. The ingredient information is information indicating an ingredient of the medicine medicated to the patient in the previously performed surgery. FIG. 2 only illustrates a part of the past auxiliary information for convenience of illustration.

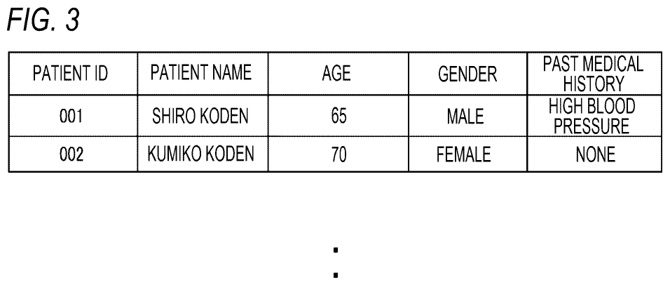

As illustrated in FIG. 3, the patient information can include age information indicating age of the patient, gender information indicating gender of the patient, and past medical history information indicating a past medical history of the patient. For example, the age information on Shiro Koden is information indicating that age of Shiro Koden is 65 years old. For example, the gender information on Shiro Koden is information indicating that Shiro Koden is male. For example, the past medical history information on Shiro Koden is information indicating that Shiro Koden has a past medical history of high blood pressure.

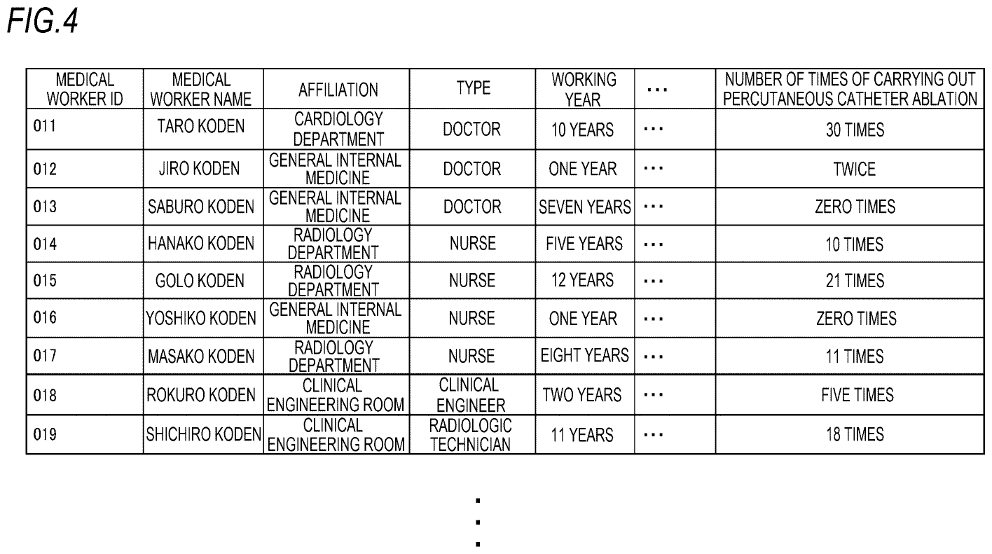

As illustrated in FIG. 4, the medical worker information can include affiliation information indicating affiliation of a medical worker, type information indicating a type of the medical worker, and experience information on surgical experiences of the medical worker. The affiliation of the medical worker can include, for example, cardiology department, general internal medicine, and clinical engineering room. The type of the medical worker is, for example, doctor and nurse. The experience information can include, for example, working year information indicating a working year of a target medical worker and experience count information indicating the number of times the target medical worker performs a target surgery. For example, the affiliation information on Taro Koden is information indicating that the affiliation of Taro Koden is cardiology department. For example, the type information on Taro Koden is information indicating that the type of Taro Koden is doctor. For example, the working year information on Taro Koden is information indicating that the number of working years of Taro Koden is 10 years. For example, the experience count information on percutaneous catheter ablation of Taro Koden is information indicating that Taro Koden performed catheter ablation 30 times.

The medical assistance device 30 will be described referring back to FIG. 1. The medical assistance device 30 is installed in a medical facility such as a hospital. The medical assistance device 30 is, for example, a patient monitor including a general server and a display. When the medical assistance device 30 is a patient monitor, the medical assistance device 30 is placed in an operating room or the like. When the medical assistance device 30 is a patient monitor, the medical assistance device 30 can display, for example, physiological information (physiological waveform information or the like) and information indicating a progress of a surgery (progress information or the like). In this case, the medical assistance device 30 may switch between display of the physiological information and display of the information indicating the progress of the surgery. The medical assistance device 30 can include an input and output interface 31 and a controller 32. The input and output interface 31 and the controller 32 are communicably connected via a bus 33.

The input and output interface 31 functions as an interface between the communication device 10 and the medical assistance device 30. The input and output interface 31 further functions as an interface between the storage device 20 and the medical assistance device 30. The input and output interface 31 further functions as an interface between the medical assistance device 30 and the user terminal device 40.

The controller 32 may have a hardware configuration the same as or similar to that of the controller 11 of the communication device 10. The controller 32 can include at least one processor 321 and a memory 322 that stores at least one instruction that can be executed by the processor 321.

In the present embodiment, a computer readable medium may be used. The computer readable medium refers to a type of physical memory (RAM, ROM, or the like) that may store information and data readable by the processor. The computer readable medium may store instructions related to execution processing by one or more processors. The term “computer readable medium” can include a tangible item and excludes a carrier wave or a temporary signal (that is, refers to a non-transitory medium). Examples of the non-transitory computer readable medium can include a magnetic recording medium (for example, a flexible disk, a magnetic tape, or a hard disk drive), a magneto-optical recording medium (for example, a magneto-optical disk), a CD read only memory (CD-ROM), a CD-R, a CD-R/W, and a semiconductor memory (for example, a mask ROM, a programmable ROM (PROM), an erasable PROM (EPROM), or a flash ROM).

The controller 32 obtains start time information indicating surgery start time. For example, when a user inputs the start time information to the user terminal device 40, the input information is transmitted from the user terminal device 40 to the medical assistance device 30. In this manner, the medical assistance device 30 obtains the start time information.

The controller 32 obtains at least one of the patient information and the medical worker information. For example, when the user inputs at least one of the patient information and the medical worker information to the user terminal device 40, the input information is transmitted from the user terminal device 40 to the medical assistance device 30. In this manner, the medical assistance device 30 obtains at least one of the patient information and the medical worker information.

For example, the controller 32 obtains past surgery information from the storage device 20 in response to a request signal from the user terminal device 40 to be described later. For example, the controller 32 obtains the past surgery information from the storage device 20 based on at least one of the patient information and the medical worker information. For example, when obtaining the past surgery information from the storage device 20 based on the patient information on Shiro Koden and the medical worker information on Taro Koden, the controller 32 obtains, from the storage device 20, past surgery information on a surgery in which the patient is Shiro Koden and Taro Koden is involved.

The controller 32 generates anticipated surgery schedule information and anticipated auxiliary information based on the obtained past surgery information. The anticipated surgery schedule information is information obtained by statistically processing the past surgery information. The anticipated surgery schedule information can include anticipated procedure schedule information indicating an anticipated procedure schedule set for each of a plurality of procedures included in a surgery.

The anticipated procedure schedule information can include anticipated required time information indicating time required for each procedure included in the currently performed surgery and order information indicating an order of each procedure included in the currently performed surgery in the entire surgery. The anticipated required time information is generated by the controller 32 based on past required time information. In the present embodiment, the anticipated required time information is a median value of time required for a procedure included in the previously performed surgery. The anticipated required time information may also be, for example, an average value of the time required for the procedure included in the previously performed surgery. Further, the controller 32 generates anticipated required total time information based on anticipated required time information on each procedure. For example, the controller 32 generates the anticipated required total time information by adding up the required time indicated by the anticipated required time information on each procedure.

The anticipated auxiliary information can include at least one of anticipated medication information and anticipated transition information. The anticipated medication information can include anticipated timing information, anticipated content information, anticipated dose information, and anticipated ingredient information. The anticipated timing information is information indicating a timing scheduled to medicate a patient in the currently performed surgery. The anticipated content information is information indicating a content of medicine scheduled to be medicated to the patient in the currently performed surgery. The anticipated dose information is information indicating a dose of the medicine scheduled to be medicated to the patient in the currently performed surgery. The anticipated ingredient information is information indicating an ingredient of the medicine scheduled to be medicated to the patient in the currently performed surgery.

The anticipated transition information is information indicating an anticipated transition of patient physiological information in the currently performed surgery. The anticipated transition information can include anticipated abnormality time information indicating time when a value indicated by the patient physiological information is an abnormal value and abnormality type information indicating a type of abnormality of the patient at the time indicated by the anticipated abnormality time information. Examples of the type of abnormality of the patient can include apnea, bradycardia, and tachycardia. Apnea means that respiration is temporarily stopped for 10 seconds or longer.

The controller 32 may obtain a plurality of pieces of medical worker information indicating a medical worker involved in a surgery corresponding to the anticipated surgery schedule information, including type information indicating a type of the medical worker. In this case, the controller 32 generates sub-anticipated surgery schedule information for each medical worker based on the obtained type information. The sub-anticipated surgery schedule information can include sub-anticipated procedure schedule information on each procedure for each medical worker. The sub-anticipated procedure schedule information can include sub-anticipated required time information and sub-order information. The sub-anticipated required time information is information indicating time required for a procedure performed by a medical worker of a target type among procedures included in the currently performed surgery. The sub-order information is information indicating an order of a procedure performed by the medical worker of the target type among the procedures included in the currently performed surgery.

When the controller 11 of the communication device 10 generates progress information, the controller 32 obtains current procedure information and the progress information, or the progress information from the communication device 10. For example, the controller 32 obtains the progress information from the communication device 10 at least at a timing when a procedure included in the currently performed surgery is completed. On the other hand, when the controller 11 of the communication device 10 does not generate the progress information, the controller 32 obtains the current procedure information from the communication device 10. The controller 32 generates progress information based on the obtained current procedure information. A method for generating progress information by the controller 32 may be the same as or similar to a method for generating progress information by the controller 11 of the communication device 10.

The controller 32 generates display data including anticipated surgery schedule information, anticipated auxiliary information, progress information, and sub-anticipated surgery schedule information. The controller 32 transmits the generated display data to the user terminal device 40.

The user terminal device 40 is a device operated by a medical worker such as a doctor or a nurse. The user terminal device 40 is an electronic device such as a desktop PC, a notebook PC, a tablet terminal, and a smartphone. The user terminal device 40 can include an input and output interface 41, an operation unit 42, and a display 43. The input and output interface 41, the operation unit 42, and the display 43 are communicably connected via a bus 44.

The input and output interface 41 functions as an interface between the medical assistance device 30 and the user terminal device 40.

Upon receiving an input operation of a user operating the user terminal device 40, the operation unit 42 generates a request signal corresponding to the input operation. The operation unit 42 is a touch panel superimposed on the display 43, an operation button attached to a case of the user terminal device 40, and the like. For example, the operation unit 42 receives an input operation for obtaining display data and generates a request signal corresponding to the input operation. The generated request signal is transmitted to the medical assistance device 30 via the input and output interface 41. The medical assistance device 30 that receives the request signal generated by the operation unit 42 generates display data based on the request signal, and transmits the generated display data to the user terminal device 40.

The display 43 is a touch screen display such as a liquid crystal display and an organic EL display. The display 43 displays, for example, an image based on the display data received from the medical assistance device 30.

### First Example

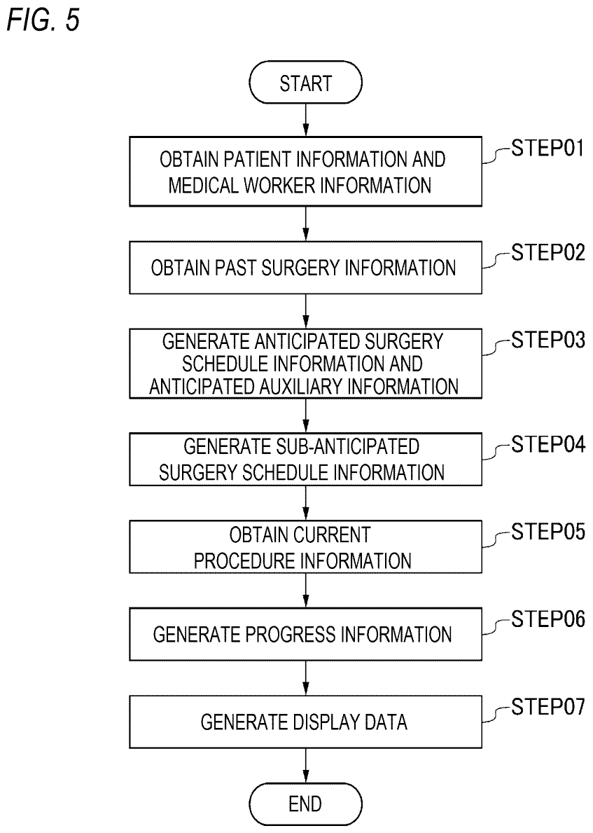

Next, a medical assistance method executed by the medical assistance device 30 according to a first example will be described with reference to FIGS. 5 to 8. In the present example, it is assumed that percutaneous catheter ablation (example of a surgery) is performed from 9:00 on Aug. 1, 2023, and current time is 9:32. In this surgery, a patient is Shiro Koden, and medical workers involved in the surgery are Taro Koden, Jiro Koden, Hanako Koden, Yoshiko Koden, Rokuro Koden, and Shichiro Koden. In the present example, it is further assumed that the medical assistance device 30 obtains current procedure information from the communication device 10 during the surgery and generates progress information based on the obtained current procedure information. The same applies to following examples unless otherwise specified.

FIG. 5 is a flowchart illustrating the medical assistance method according to the first example. As illustrated in FIG. 5, the controller 32 of the medical assistance device 30 obtains patient information and medical worker information (STEP 01). In the present example, the patient information and the medical worker information are input to the user terminal device 40 by a user and transmitted from the user terminal device 40 to the medical assistance device 30, so that the controller 32 obtains the patient information and the medical worker information. In the present example, the controller 32 obtains the patient information on Shiro Koden, and the medical worker information on Taro Koden, Jiro Koden, Hanako Koden, Yoshiko Koden, Rokuro Koden, and Shichiro Koden.

The controller 32 obtains past surgery information from the storage device 20 based on the obtained patient information and medical worker information (STEP 02). In the present example, the controller 32 obtains the past surgery information from the storage device 20 based on the patient information on Shiro Koden and the medical worker information on Taro Koden. Alternatively, the controller 32 may obtain the past surgery information from the storage device 20 based on, for example, the patient information on Shiro Koden and the medical worker information on a plurality of medical workers involved in the surgery. Since the controller 32 obtains the past surgery information from the storage device 20 based on the patient information on Shiro Koden and the medical worker information on Taro Koden, the controller 32 can obtain the past surgery information from the storage device 20 in consideration of a past medical history of Shiro Koden, a working year of Taro Koden, and the like.

The controller 32 generates anticipated surgery schedule information and anticipated auxiliary information based on the obtained past surgery information (STEP 03). In the present example, the controller 32 generates the anticipated surgery schedule information and the anticipated auxiliary information based on past surgery information on a surgery such as the surgery number 1 illustrated in FIG. 2.

As illustrated in FIG. 5, the controller 32 generates sub-anticipated surgery schedule information for each medical worker based on obtained type information (STEP 04). In the present example, the controller 32 generates the sub-anticipated surgery schedule information on Taro Koden, Jiro Koden, Hanako Koden, Yoshiko Koden, Rokuro Koden, and Shichiro Koden.

The controller 32 obtains current procedure information from the communication device 10 (STEP 05). In the present example, the controller 32 obtains the current procedure information from the communication device 10 at a prescribed time interval. The prescribed time interval is, for example, an interval of one minute.

The controller 32 generates progress information based on the current procedure information obtained from the communication device 10 (STEP 06). The controller 32 generates the progress information by, for example, analyzing image data captured by the communication device 10 during the surgery.

The controller 32 generates display data including the anticipated surgery schedule information, the anticipated auxiliary information, the progress information, the sub-anticipated surgery schedule information, current medication information, and current transition information (STEP 07). When the display data is generated, the controller 32 transmits the generated display data to the user terminal device 40.

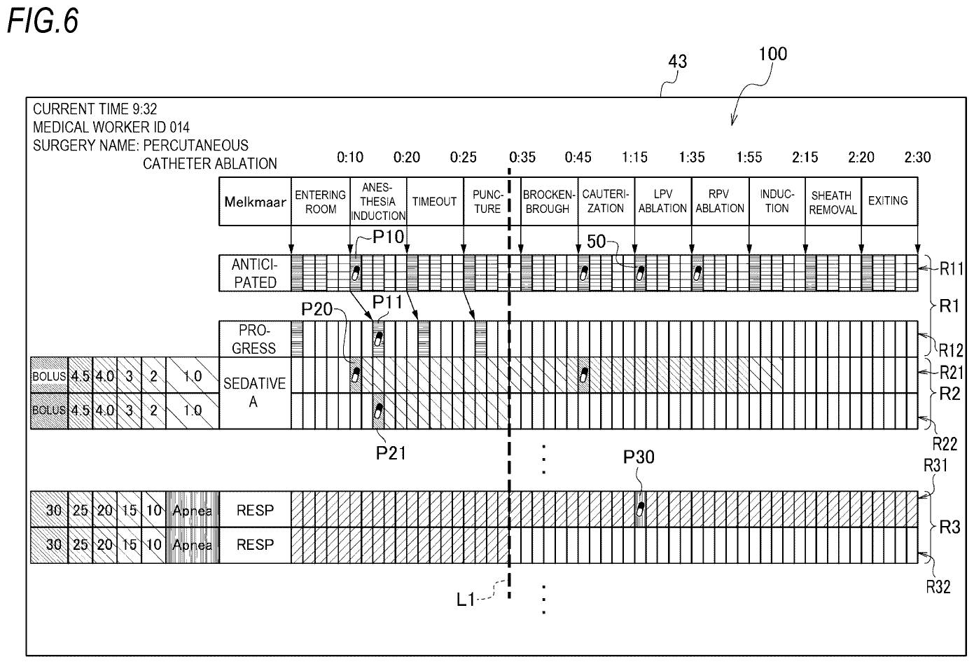

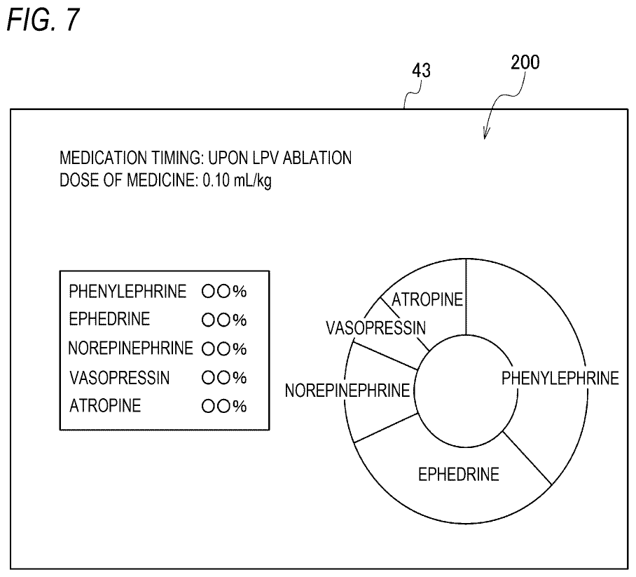

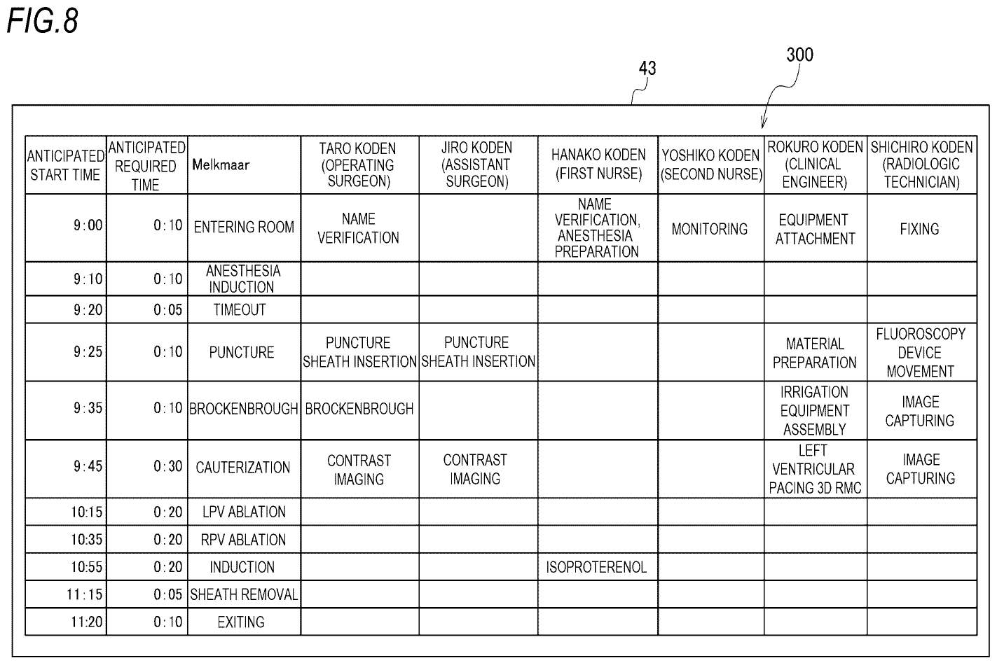

An image based on the display data received from the controller 32 of the medical assistance device 30 is displayed on the display 43 of the user terminal device 40. Examples of the image displayed on the display 43 can include an image 100 illustrated in FIG. 6, an image 200 illustrated in FIG. 7, and an image 300 illustrated in FIG. 8. Hereinafter, a case in which the images 100, 200, and 300 are displayed on the display 43 of the user terminal device 40 operated by Hanako Koden will be described as an example, and the images 100, 200, and 300 will be described. An image displayed on the display 43 of the user terminal device 40 operated by a medical worker other than Hanako Koden is basically the same as or similar to the images 100, 200, and 300 displayed on the display 43 of the user terminal device 40 operated by the Hanako Koden.

A case in which the image 100 illustrated in FIG. 6 is displayed on the display 43 will be described. The image 100 displays current time, medical worker ID, a surgery name of the currently performed surgery, a plurality of procedures included in the surgery, anticipated start time of each procedure, anticipated surgery schedule information, progress information on each procedure, anticipated medication information, current medication information, anticipated transition information, and current transition information. The anticipated start time of each procedure is calculated by the controller 32 based on, for example, start time information, order information, and anticipated required time information.

The image 100 displays a line L1 indicating a position of the current time. In the present example, the line L1 is a broken line, and may also be a solid line, a dashed line, and the like. The position of the current time is not limited to the line L1.

The image 100 contains a region R1, a region R2, and a region R3. The region R1 displays anticipated surgery schedule information including anticipated procedure schedule information on each procedure and progress information on each procedure.

The anticipated procedure schedule information and the progress information are displayed side by side in two stages. That is, the anticipated procedure schedule information on a procedure included in the currently performed surgery in association with the progress information is displayed in a vicinity of the progress information. In the present example, the anticipated procedure schedule information is displayed above the progress information.

A shade of color in the region R1 represents how much start time of each procedure deviates from a median value. In the present specification, the shade of color is represented by the number of hatch lines. A larger number of hatch lines indicates a darker color. In the present example, a part with a darkest color is the median value of the start time in the procedure, and as the color becomes lighter, the start time deviates further from the median value. In the region R1, a part with a darkest color in an upper region (that is, a region indicating an anticipated schedule of each procedure) R11 indicates a position of time corresponding to a median value of anticipated start time of a procedure. For example, a part P10 in FIG. 6 represents a position of time corresponding to a median value of anticipated start time of a procedure of anesthesia induction. In a lower region (that is, a region representing a progress of each procedure) R12 of the region R1, a part with the same color as the part with the darkest color in the region R11 represents a position of time when the procedure is started. For example, a part P11 in FIG. 6 represents a position of time when the procedure of anesthesia induction is started. In this manner, a part representing a position of time corresponding to a median value of anticipated start time of a certain procedure has the same color as a part representing a position of time when the procedure is started, and thus, for example, a medical worker can easily and visually associate these times.

Anticipated medication information on a sedative A is displayed in an upper region R21 of the region R2, and current medication information on the sedative A is displayed in a lower region R22 of the region R2. A shade of color in the region R2 represents a dose of the sedative A medicated to the patient, and a darker color indicates a larger dose of the sedative A medicated to the patient. A darkest part in the upper region R21 of the region R2 represents scheduled time when the sedative A bolus is administered. For example, a part P20 in FIG. 6 represents scheduled time when the sedative A bolus is administered in the procedure of anesthesia induction. In the lower region R22 of the region R2, a part with a darkest color represents time when the sedative A bolus is administered to the patient. For example, a part P21 in FIG. 6 represents time when the sedative A bolus is administered to the patient in the procedure of anesthesia induction.

Anticipated transition information is displayed in an upper region R31 of the region R3, and current transition information is displayed in a lower region R32 of the region R3. A shade of color in the region R3 represents a patient condition level corresponding to a value indicated by patient physiological information. A part (for example, a part P30) hatched by vertical lines indicates time when the value indicated by the patient physiological information is an abnormal value. The region R3 further displays a type of abnormality of the patient at the time when the value indicated by the patient physiological information is an abnormal value. In the present example, apnea is displayed as the type of abnormality of the patient.

Next, the image 200 illustrated in FIG. 7 will be described. For example, when the medical worker clicks an icon 50 included in the image 100 illustrated in FIG. 6, the image 200 illustrated in FIG. 7 is displayed on the display 43 of the user terminal device 40. In the present example, the icon 50 is an icon imitating a medicine capsule, and may also be an icon of another design. A character or the like may be displayed instead of the icon 50. As illustrated in FIG. 7, the image 200 contains anticipated timing information, anticipated dose information, and anticipated ingredient information on a medicine corresponding to the icon 50. For this reason, by visually checking the image 200, the medical worker can recognize a medication timing of the medicine corresponding to the icon 50, a dose of the medicine, and an ingredient of the medicine.

Next, the image 300 illustrated in FIG. 8 will be described. For example, when the medical worker performs a prescribed operation on the operation unit 42 of the user terminal device 40 to display the image 300 on the display 43, the image 300 is displayed on the display 43. The image 300 can include a sub-anticipated surgery schedule of each medical worker. The sub-anticipated surgery schedule is based on sub-anticipated surgery schedule information on a corresponding medical worker. In the present example, the image 300 can include sub-anticipated surgery schedules of Taro Koden, Jiro Koden, Hanako Koden, Yoshiko Koden, Rokuro Kode, and Shichiro Koden. The display 43 may also display an image including the sub-anticipated surgery schedule of at least one of Taro Koden, Jiro Koden, Hanako Koden, Yoshiko Koden, Rokuro Koden, and Shichiro Koden. That is, the image displayed on the display 43 does not necessarily contain the sub-anticipated surgery schedules of all the medical workers involved in the surgery.

By visually checking the image 300, the medical worker can recognize his or her sub-anticipated surgery schedule and the sub-anticipated surgery schedule of another medical worker. For example, the medical worker can recognize a scheduled procedure to be performed by himself or herself and anticipated start time of the procedure in the currently performed surgery by visually checking his or her sub-anticipated surgery schedule.

The medical assistance device 30 having the above-described configuration generates display data including anticipated surgery schedule information and anticipated auxiliary information, the anticipated auxiliary information including anticipated medication information and anticipated transition information, based on past surgery information including past required total time information and past auxiliary information including medication information and transition information. For this reason, a medical worker can recognize not only the anticipated surgery schedule information but also the anticipated medication information and the anticipated transition information by visually checking the display data. Accordingly, the medical assistance device 30 can assist even a less experienced medical worker to appropriately perform a surgery.

The medical assistance device 30 having the above-described configuration generates display data including anticipated surgery schedule information and anticipated medication information based on past surgery information including medication information including timing information, content information, and dose information. Since the medication information can include not only timing information but also content information and dose information, the medical worker can recognize not only a medication timing but also a content and a dose of medicine to be medicated by visually checking the display data.

According to the medical assistance device 30 having the above-described configuration, the anticipated transition information can include anticipated abnormality time information indicating time when a value indicated by patient physiological information is an abnormal value. For this reason, even a less experienced medical worker can appropriately perform a surgery while recognizing the time when the value indicated by the patient physiological information is an abnormal value.

According to the medical assistance device 30 having the above-described configuration, the anticipated transition information can include abnormality type information indicating a type of abnormality of the patient at the time indicated by the anticipated abnormality time information. For this reason, even a less experienced medical worker can appropriately perform a surgery while recognizing the type of abnormality of the patient at the time indicated by the anticipated abnormality time information.

According to the medical assistance device 30 having the above-described configuration, the past surgery information is obtained based on patient information and medical worker information, and thus the anticipated surgery schedule information generated based on the past surgery information reflects characteristics of the patient and the medical worker. For this reason, the medical assistance device 30 can generate highly accurate anticipated surgery schedule information.

According to the medical assistance device 30 having the above-described configuration, the patient information can include age information, gender information, and past medical history information, and thus more accurate anticipated surgery schedule information can be generated.

According to the medical assistance device 30 having the above-described configuration, the medical worker information can include affiliation information, type information, and experience information, and thus more accurate anticipated surgery schedule information can be generated.

According to the medical assistance device 30 having the above-described configuration, the display data can include sub-anticipated surgery schedule information on each medical worker generated based on the type information indicating a type of a medical worker. For this reason, each medical worker can perform an appropriate procedure at an appropriate timing by, for example, visually checking the sub-anticipated surgery schedule information included in the display data.

According to the medical assistance device 30 having the above-described configuration, the anticipated surgery schedule information can include anticipated auxiliary information and anticipated required time information, and thus the medical worker can recognize a timing of medication, a transition of the patient physiological information, and time required for each procedure in association with each procedure. Accordingly, the medical assistance device 30 can assist even a less experienced medical worker to more appropriately perform a surgery.

According to the medical assistance device 30 having the above-described configuration, the anticipated required time information is a median value of time required for a procedure included in the previously performed surgery, and thus the anticipated required time information can be information indicating more standardized anticipated required time.

### Second Example

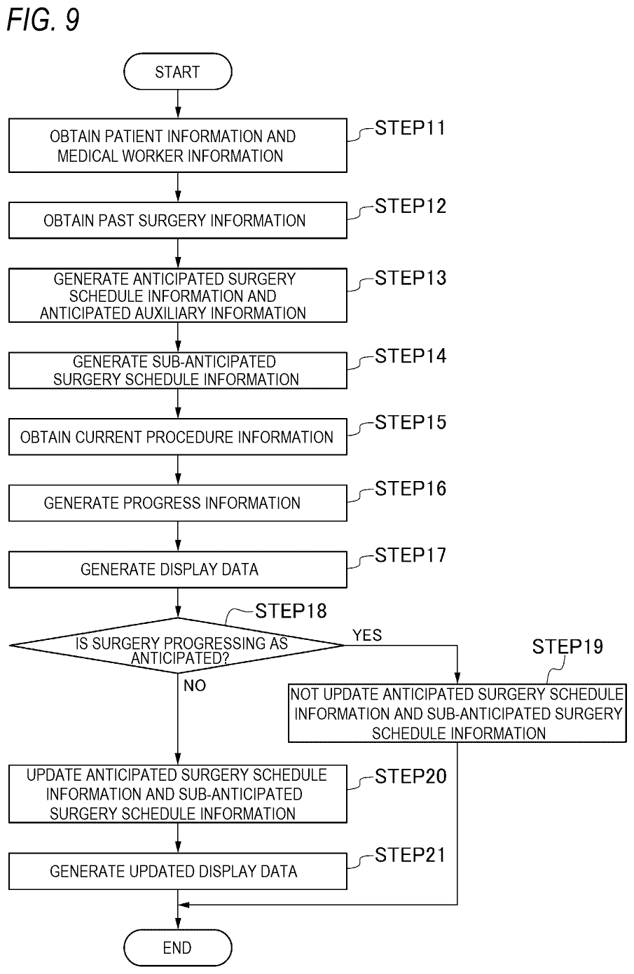

Next, a medical assistance method executed by the medical assistance device 30 according to a second example will be described with reference to FIGS. 9 to 11. In the second example, parts the same or similar to those of the first example are denoted by the same or similar reference numerals as those of the first example, and description of repeated parts is omitted as appropriate. The present example is different from the first example in that anticipated surgery schedule information and sub-anticipated surgery schedule information are updated to reflect delays and advances of procedures performed up to current time among a plurality of procedures included in a surgery. In the present example, it is assumed that a delay of four minutes occurs in entering the room, an advance of three minutes occurs in anesthesia induction, and a delay of one minute occurs in timeout.

In the present example, the controller 32 can determine whether the currently performed surgery is progressing as anticipated based on progress information and anticipated procedure schedule information. For example, the controller 32 determines whether the currently performed surgery is progressing as anticipated based on time of determining that a currently performed procedure is completed and anticipated completion time calculated based on the anticipated procedure schedule information and start time information. The anticipated completion time is estimated time when a procedure is completed. When determining that the currently performed surgery is progressing as anticipated, the controller 32 does not update the anticipated surgery schedule information and the sub-anticipated surgery schedule information. On the other hand, when determining that the currently performed surgery is not progressing as anticipated, the controller 32 updates the anticipated surgery schedule information and the sub-anticipated surgery schedule information at a prescribed timing based on the progress information. In the present example, the controller 32 updates the anticipated surgery schedule information and the sub-anticipated surgery schedule information when each procedure is completed. The controller 32 may also update the anticipated surgery schedule information and the sub-anticipated surgery schedule information, for example, when each procedure is started, or periodically update the anticipated surgery schedule information and the sub-anticipated surgery schedule information at a prescribed time interval.

When the anticipated surgery schedule information and the sub-anticipated surgery schedule information are updated, the controller 32 generates updated display data (example of display data) based on the updated anticipated surgery schedule information and sub-anticipated surgery schedule information. The updated display data can include updated anticipated surgery schedule information, updated sub-anticipated surgery schedule information, anticipated auxiliary information, progress information, current medication information, and current transition information. The controller 32 transmits the updated display data to the user terminal device 40.

Next, a medical assistance method executed by the medical assistance device 30 according to the present example will be described with reference to FIG. 9. STEP 11 to STEP 17 are the same as STEP 01 to STEP 07 of the first example.

The controller 32 determines whether a currently performed surgery is progressing as anticipated based on progress information and anticipated procedure schedule information (STEP 18). When determining that the currently performed surgery is progressing as anticipated (YES in STEP 18), the controller 32 does not update anticipated surgery schedule information and sub-anticipated surgery schedule information (STEP 19). On the other hand, when determining that the currently performed surgery is not progressing as anticipated (NO in STEP 18), the controller 32 updates the anticipated surgery schedule information and the sub-anticipated surgery schedule information when each procedure is completed based on progress information (STEP 20).

In the present example, a delay of four minutes occurs in entering the room, an advance of three minutes occurs in anesthesia induction, and a delay of one minute occurs in timeout. For this reason, the controller 32 updates the anticipated surgery schedule information and the sub-anticipated surgery schedule information when each procedure is completed based on progress information on each procedure in procedures including entering the room, anesthesia induction, and timeout. Accordingly, the delay or advance in each procedure is reflected in the anticipated surgery schedule information and the sub-anticipated surgery schedule information.

When the anticipated surgery schedule information and the sub-anticipated surgery schedule information are updated, the controller 32 generates updated display data based on the updated anticipated surgery schedule information and sub-anticipated surgery schedule information (STEP 21). When the updated display data is generated, the controller 32 transmits the generated updated display data to the user terminal device 40.

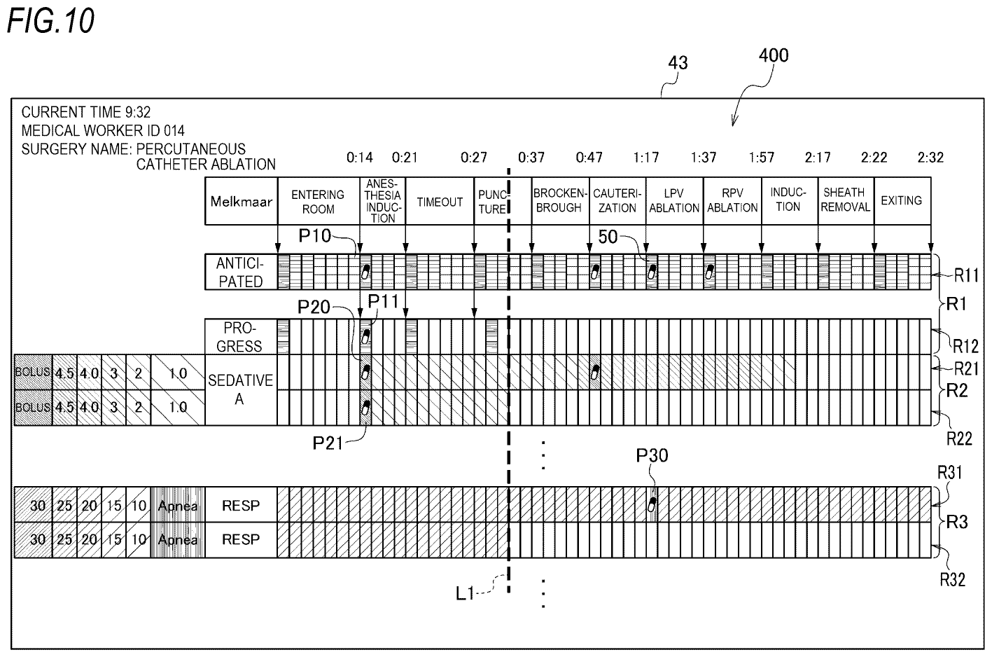

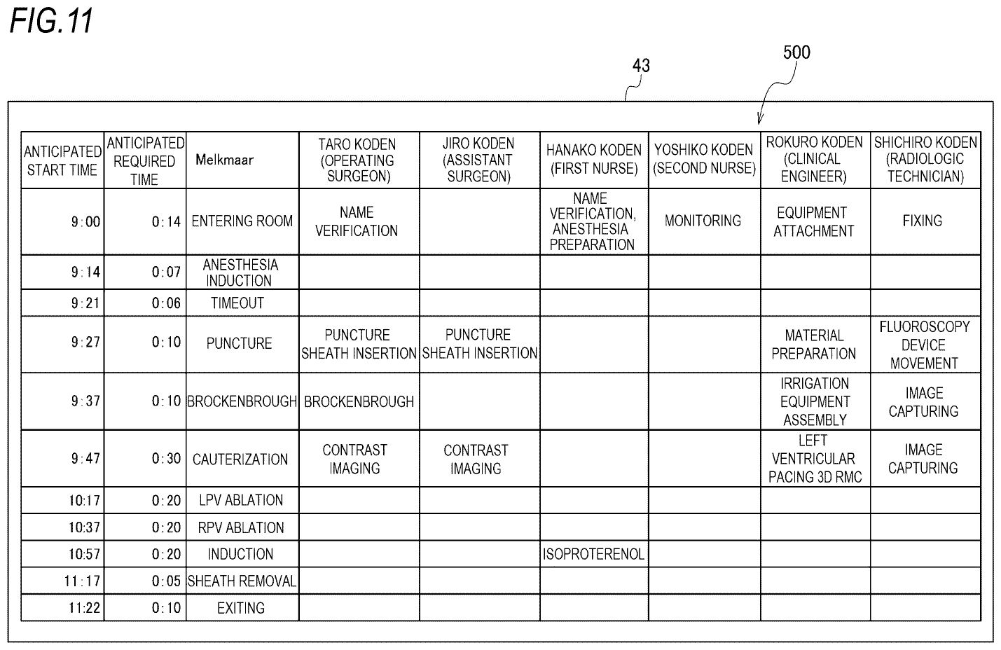

When the user terminal device 40 receives the updated display data from the medical assistance device 30, an image based on the updated display data is displayed on the display 43 of the user terminal device 40. The image displayed on the display 43 is, for example, the image 200 illustrated in FIG. 7, an image 400 illustrated in FIG. 10, and an image 500 illustrated in FIG. 11.

A case in which the image 400 illustrated in FIG. 10 is displayed on the display 43 will be described. As illustrated in FIG. 10, in procedures from entering the room to puncture in the region R1 to the region R3 in the image 400, squares are added or deleted based on a delay or an advance occurred until current time. In the region R1 to the region R3, completion time of each procedure from entering the room to puncture is updated based on a delay or an advance occurred until the current time. The completion time is time when a procedure is completed. In the image 400, anticipated start time of each procedure and anticipated completion time of each procedure are updated based on a delay (in the present example, a delay of two minutes) occurred until the current time in procedures after Brockenbrough in the region R1 to the region R3. The image 400 is different from the image 100 according to the first example in these aspects. Other aspects are the same or similar in the image 400 and the image 100 according to the first example.

Next, the image 500 illustrated in FIG. 11 will be described. For example, when a medical worker performs a prescribed operation on the operation unit 42 of the user terminal device 40 to display the image 500 on the display 43, the image 500 is displayed on the display 43. The image 500 can include an updated sub-anticipated surgery schedule for each medical worker. The updated sub-anticipated surgery schedule is based on updated sub-anticipated surgery schedule information on a corresponding medical worker. As illustrated in FIG. 11, the image 500 is different from the image 300 according to the first example in that the image 500 can include anticipated start time and anticipated required time that reflect a delay or an advance of each procedure performed until the current time among a plurality of procedures included in the surgery.

Also in the present example, the medical assistance device 30 has effects the same as or similar to those of the first example.

### Third Example

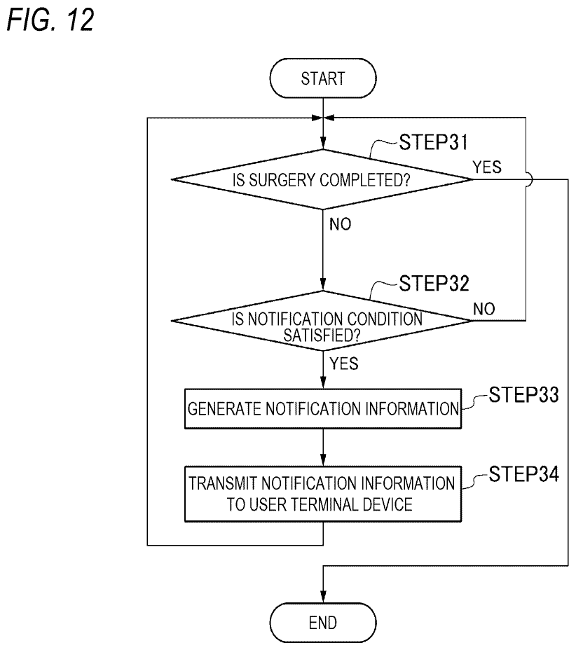

Next, a notification method executed by the medical assistance device 30 according to a third example will be described with reference to FIG. 12. FIG. 12 is a flowchart illustrating the notification method according to the third example. In the third example, parts the same as or similar to those of the first example or the second example are denoted by the same or similar reference numerals as those of the first example or the second example, and description of repeated parts is omitted as appropriate. The third example is different from the first example or the second example in that the medical assistance device 30 executes not only the medical assistance method according to the first example or the second example but also the notification method illustrated in FIG. 12. The notification method illustrated in FIG. 12 is executed during a period from a start to an end of a surgery.

In the present example, the controller 32 can determine whether the currently performed surgery is completed based on progress information on a last procedure among a plurality of procedures included in the currently performed surgery.

The controller 32 notifies a medical worker scheduled to perform an upcoming procedure of notification information according to a progress of a procedure before the upcoming procedure. In the present example, the controller 32 determines whether a prescribed notification condition is satisfied, generates the notification information when determining that the notification condition is satisfied, and transmits the generated notification information to the user terminal device 40 to perform notification of the notification information. In the present example, the prescribed notification condition is whether a procedure two steps before the upcoming procedure is completed. The prescribed notification condition may also be, for example, whether a procedure immediately before the upcoming procedure is started. The notification method for the notification information may be an auditory method or a visual method.

As illustrated in FIG. 12, the controller 32 determines whether the currently performed surgery is completed based on progress information on a last procedure among a plurality of procedures included in the currently performed surgery (STEP 31). When determining that the currently performed surgery is completed (YES in STEP 31), the controller 32 ends the process according to the notification method. On the other hand, when determining that the currently performed surgery is not completed (NO in STEP 31), the controller 32 executes STEP 32.

The controller 32 determines whether a prescribed notification condition (that is, a condition as to whether a procedure immediately before an upcoming procedure is completed) is satisfied (STEP 32). When determining that the prescribed notification condition is satisfied (YES in STEP 32), the controller 32 generates notification information (STEP 33). Then, the controller 32 transmits the generated notification information to the user terminal device 40 (STEP 34). When the user terminal device 40 receives the notification information from the controller 32 of the medical assistance device 30, the display 43 of the user terminal device 40 displays, for example, character information based on the received notification information. The character information is a message such as “the procedure scheduled to be performed by you is upcoming”. By visually checking the character information displayed on the display 43 of the user terminal device 40, a medical worker can recognize that the procedure scheduled to be performed by himself or herself is upcoming. When STEP 34 is executed, the controller 32 executes STEP 31 again. On the other hand, when determining that the prescribed notification condition is not satisfied (NO in STEP 32), the controller 32 executes STEP 31 again without executing STEP 33 and STEP 34.

In this manner, the controller 32 executes the notification method illustrated in FIG. 12 from the start to the end of the surgery, and ends the notification method when the surgery is completed.

Also in the present example, the medical assistance device 30 has effects the same as or similar to those of the first example and the second example.

The above-described embodiment is intended to facilitate understanding of the presently disclosed subject matter and does not limit the presently disclosed subject matter. The presently disclosed subject matter can be changed or improved without departing from the gist thereof.

In the above-described embodiment, the controller 32 may correct anticipated procedure schedule information based on total required time, which is a total of required time indicated by past required time information, and time required for the entire previously performed surgery. For example, when the total required time is three hours and the time required for the entire previously performed surgery is two hours 30 minutes, the controller 32 multiplies time required for each procedure included in the currently performed surgery by 5/6 (150 minutes/180 minutes). That is, in this case, the controller 32 corrects the anticipated procedure schedule information by setting the required time indicated by the anticipated required time information for each procedure to be 5/6 times thereof. According to the medical assistance device 30 having such a configuration, the anticipated procedure schedule information is corrected based on the total required time and the time required for the entire previously performed surgery. Accordingly, more appropriate anticipated procedure schedule information can be obtained.

In the above-described embodiment, the medical assistance device 30 may include a storage device the same as or similar to the storage device 20 as indicated by a broken line in FIG. 1. In this case, the medical assistance system 1 may not include the storage device 20.

In the above-described embodiment, the medical assistance device 30 may include a display 35 the same as or similar to the display 43 of the user terminal device 40 as indicated by an alternate long and short dash line in FIG. 1. In this case, the images 100, 200, 300, 400, and 500 may be displayed on the display 35 of the medical assistance device 30.

In the above-described embodiment, the controller 32 generates anticipated surgery schedule information and anticipated auxiliary information based on obtained past surgery information, and the presently disclosed subject matter is not limited to this. For example, the controller 32 may generate the anticipated surgery schedule information and the anticipated auxiliary information based on schedule information indicating a schedule uniquely determined by a medical facility, a medical worker, and the like and auxiliary information indicating a medication timing or the like uniquely determined by a medical facility, a medical worker, and the like. The controller 32 may also generate the anticipated surgery schedule information and the anticipated auxiliary information based on schedule information indicating a schedule recommended by medical societies and auxiliary information indicating a medication timing or the like recommended by medical societies.

In the above-described embodiment, past auxiliary information can include medication information and transition information, and may also include only one of the medication information and the transition information.

In the above-described embodiment, medication information can include timing information, content information, dose information, and ingredient information, and may also include at least the timing information. The medication information may include at least one of the timing information, the content information, and the dose information.

In the above-described embodiment, medical worker information can include affiliation information, type information, and experience information, and may not include all of this information. That is, the medical worker information may include at least one of the affiliation information, the type information, and the experience information.

In the above-described embodiment, patient information can include age information, gender information, and past medical history information, and may not include all of this information. That is, the patient information may include at least one of the age information, the gender information, and the past medical history information.

In the above-described embodiment, the controller 32 of the medical assistance device 30 obtains past surgery information from the storage device 20 based on patient information and medical worker information, and the presently disclosed subject matter is not limited thereto. For example, the controller 32 may obtain the past surgery information from the storage device 20 based on either the patient information or the medical worker information. The controller 32 may obtain all the past surgery information from the storage device 20.

In the above-described embodiment, the controller 32 generates display data including anticipated surgery schedule information, anticipated auxiliary information, progress information, sub-anticipated surgery schedule information, current medication information, and current transition information, and the presently disclosed subject matter is not limited thereto. For example, the display data may not include the anticipated auxiliary information or the progress information. Further, the display data generated by the controller 32 may not include at least one of the sub-anticipated surgery schedule information, the current medication information, and the current transition information.

In the above-described embodiment, the controller 32 of the medical assistance device 30 generates progress information. Alternatively, for example, the controller 11 of the communication device 10 may generate the progress information. In this case, the communication device 10 transmits the progress information, or current procedure information and the progress information, to the medical assistance device 30. In this case, since the medical assistance device 30 obtains the progress information from the communication device 10, a user such as a medical worker does not necessarily input progress information into the medical assistance device 30, and as a result, a burden on the user can be reduced. In this case, the medical assistance device 30 may obtain the progress information from the communication device 10 at least at a timing, for example, when a procedure included in the currently performed surgery is completed. In this case, since the progress information is obtained from the communication device 10 at least at the timing when a procedure included in the currently performed surgery is completed, it is possible to more accurately assist in appropriately performing procedures included in the surgery.

In the above-described embodiment, the medical assistance device 30 obtains current procedure information from the communication device 10, and the presently disclosed subject matter is not limited thereto. For example, the user may input the current procedure information to the user terminal device 40, and the input current procedure information may be transmitted from the user terminal device 40 to the medical assistance device 30, so that the medical assistance device 30 may obtain the current procedure information.

In the above-described embodiment, in a vicinity of progress information, anticipated procedure schedule information on a procedure included in the currently performed surgery in association with the progress information is displayed. Alternatively, the anticipated procedure schedule information may not be displayed in the vicinity of the progress information.

In the above-described embodiment, anticipated transition information can include anticipated abnormality time information, and may not include the anticipated abnormality time information. The anticipated transition information can include abnormality type information, and may not include the abnormality type information.

In the above-described embodiment, the image 100 or the image 400, the image 200, and the image 300 or the image 500 are individually displayed on the display 43 of the user terminal device 40, and the presently disclosed subject matter is not limited thereto. For example, two images (for example, the image 100 and the image 200) or three images (for example, the image 100, the image 200, and the image 300) among these images may be displayed on the display 43 at the same time. For example, when a user such as a medical worker taps on a melkmaar region illustrated in FIG. 6, a pop-up or the like for selecting a medical worker is displayed. Then, when a medical worker is freely selected by the user from the displayed pop-up or the like, a sub-anticipated surgery schedule related to the selected medical worker is inserted and displayed in the image 100 (for example, an upper side of a landmark illustrated in FIG. 6).

In the third example, a notification method for notification information is a visual method, and may also be, for example, an auditory method, or an auditory method and a visual method.

As described above, the present specification discloses following matters.
(1) A medical assistance device including:
   at least one processor; and
   at least one memory that stores at least one instruction executable by the processor, wherein
   when the at least one instruction is executed by the processor, the medical assistance device is configured to:
   obtain past surgery information, the past surgery information including past required total time information on a previously performed surgery and past auxiliary information, the past auxiliary information including at least one of medication information, the medication information including at least timing information indicating a timing of medicating a patient in the previously performed surgery, and transition information indicating a transition of patient physiological information in the previously performed surgery;
   generate anticipated surgery schedule information and anticipated auxiliary information, the anticipated auxiliary information including at least one of anticipated medication information and anticipated transition information, based on the past surgery information; and
   generate display data including the anticipated surgery schedule information and the anticipated auxiliary information.
(2) The medical assistance device according to (1), wherein
   the past surgery information includes the medication information,
   the medication information includes the timing information and at least one of content information indicating a content of medicine medicated to the patient in the previously performed surgery and dose information indicating a dose of the medicine, and
   the display data includes the anticipated surgery schedule information and the anticipated medication information generated based on the medication information.
(3) The medical assistance device according to (1) or (2), wherein
   the display data includes the anticipated transition information, and
   the anticipated transition information includes anticipated abnormality time information indicating time when a value indicated by the physiological information is an abnormal value.
(4) The medical assistance device according to (3), wherein the anticipated transition information includes abnormality type information indicating a type of abnormality of the patient at the time indicated by the anticipated abnormality time information.
(5) The medical assistance device according to any one of (1) to (4), wherein the past surgery information is obtained based on at least one of patient information indicating a patient subjected to a surgery corresponding to the anticipated surgery schedule information and medical worker information indicating a medical worker involved in the surgery.
(6) The medical assistance device according to (5), wherein the patient information includes at least one of age information indicating age of the patient, gender information indicating gender of the patient, and past medical history information indicating a past medical history of the patient.
(7) The medical assistance device according to (5) or (6), wherein the medical worker information includes at least one of affiliation information indicating affiliation of the medical worker, type information indicating a type of the medical worker, and experience information on a currently performed surgery by the medical worker.
(8) The medical assistance device according to any one of (1) to (7), wherein
   the medical assistance device is configured to
      obtain a plurality of pieces of medical worker information indicating a medical worker involved in a surgery corresponding to the anticipated surgery schedule information, the medical worker information including type information indicating a type of the medical worker, and
      generate sub-anticipated surgery schedule information for each medical worker based on the type information, and
   the display data includes the sub-anticipated surgery schedule information.
(9) The medical assistance device according to any one of (1) to (8), wherein
   the past surgery information includes past required time information indicating time required for each procedure included in the previously performed surgery,
   the medical assistance device is configured to generate anticipated required time information indicating time required for the procedure based on the past required time information, and
   the anticipated surgery schedule information includes the anticipated required time information on each procedure corresponding to the anticipated surgery schedule information.
(10) The medical assistance device according to (9), wherein the anticipated required time information is a median value of time required for a procedure included in the previously performed surgery.

1. A medical assistance device comprising:
at least one processor; and
at least one memory that stores at least one instruction executable by the processor, wherein
when the at least one instruction is executed by the processor, the medical assistance device is configured to:
obtain past surgery information, the past surgery information including past required total time information on a previously performed surgery and past auxiliary information, the past auxiliary information including at least one of medication information, the medication information including at least timing information indicating a timing of medicating a patient in the previously performed surgery, and transition information indicating a transition of patient physiological information in the previously performed surgery;
generate anticipated surgery schedule information and anticipated auxiliary information, the anticipated auxiliary information including at least one of anticipated medication information and anticipated transition information, based on the past surgery information; and
generate display data including the anticipated surgery schedule information and the anticipated auxiliary information.

2. The medical assistance device according to claim 1, wherein
the past surgery information includes the medication information,
the medication information includes the timing information and at least one of content information indicating a content of medicine medicated to the patient in the previously performed surgery and dose information indicating a dose of the medicine, and
the display data includes the anticipated surgery schedule information and the anticipated medication information generated based on the medication information.

3. The medical assistance device according to claim 1, wherein
the display data includes the anticipated transition information, and
the anticipated transition information includes anticipated abnormality time information indicating time when a value indicated by the physiological information is an abnormal value.

4. The medical assistance device according to claim 3, wherein the anticipated transition information includes abnormality type information indicating a type of abnormality of the patient at the time indicated by the anticipated abnormality time information.

5. The medical assistance device according to claim 1, wherein the past surgery information is obtained based on at least one of patient information indicating a patient subjected to a surgery corresponding to the anticipated surgery schedule information and medical worker information indicating a medical worker involved in the surgery.

6. The medical assistance device according to claim 5, wherein the patient information includes at least one of age information indicating age of the patient, gender information indicating gender of the patient, and past medical history information indicating a past medical history of the patient.

7. The medical assistance device according to claim 5, wherein the medical worker information includes at least one of affiliation information indicating affiliation of the medical worker, type information indicating a type of the medical worker, and experience information on a currently performed surgery by the medical worker.

8. The medical assistance device according to claim 1, wherein
the medical assistance device is configured to
obtain a plurality of pieces of medical worker information indicating a medical worker involved in a surgery corresponding to the anticipated surgery schedule information, the medical worker information including type information indicating a type of the medical worker, and
generate sub-anticipated surgery schedule information for each medical worker based on the type information, and
the display data includes the sub-anticipated surgery schedule information.

9. The medical assistance device according to claim 1, wherein
the past surgery information includes past required time information indicating time required for each procedure included in the previously performed surgery,
the medical assistance device is configured to generate anticipated required time information indicating time required for the procedure based on the past required time information, and
the anticipated surgery schedule information includes the anticipated required time information on each procedure corresponding to the anticipated surgery schedule information.

10. The medical assistance device according to claim 9, wherein the anticipated required time information is a median value of time required for a procedure included in the previously performed surgery.

11. The medical assistance device according to claim 1, further comprising an input and output interface, wherein
the medical assistance device obtains the past surgery information from an external storage device through the input and output interface, and
the external storage device is outside of the medical assistance device.
